(19) United States
(12) Patent Application Publication
(10) Pub. No.: US 2013/0182223 A1
(43) Pub. Date: Jul. 18, 2013

(54) SUSPENDED GONIOLENS SYSTEM

(71) Applicants: John WARDLE, San Clemente, CA (US); Gabor SCHARIOTH, Reckinghausen (DE)

(72) Inventors: John WARDLE, San Clemente, CA (US); Gabor SCHARIOTH, Reckinghausen (DE)

(21) Appl. No.: 13/744,351

(22) Filed: Jan. 17, 2013

Related U.S. Application Data
(60) Provisional application No. 61/587,250, filed on Jan. 17, 2012.

(51) Int. Cl. A61B 3/117

(52) U.S. Cl. CPC A61B 3/117
USPC 351/219; 351/246

(57) ABSTRACT

A suspended goniolens system is provided. The suspended goniolens system includes a balance arm with a goniolens disposed at one end and a counterbalance disposed towards an opposing end. The system can position the goniolens in a desired position near a patient's eye and can maintain the desired position without the need to be held in place by a clinician. The suspended goniolens system allows for the clinician to use both hands during treatment of the patient. The goniolens system can be attached to an optical microscope or an adapter engaged with the optical microscope. Methods are also provided for using the goniolens system.

### CROSS REFERENCE TO RELATED APPLICATIONS

This application claims the benefit of priority to U.S. Provisional Application Ser. No. 61/587,250 filed on Jan. 17, 2012, the disclosure of which is incorporated by reference herein.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present disclosure relates generally to a mechanical apparatus that can be used to facilitate gonioscopic surgery.

### BACKGROUND

Gonioscopy is an ophthalmology procedure that requires the use of a goniolens in addition to an operating microscope to gain a view of the anatomical angle formed between the eye's cornea and iris.

The procedure allows the clinician to view the irideocorneal angle through a mirror or prism, without which the angle is masked by total internal reflection from the ocular tissue. The importance of this process is in diagnosing and monitoring various eye conditions associated with glaucoma. Without gonioscopy, identification of the underlying mechanism and, therefore, the appropriate treatment of any glaucomatous condition is impossible. Gonioscopy is also used when performing various procedures for the treatment of glaucoma such as implantation of a drainage device, laser trabeculoplasty, peripheral laser gonioplasty, goniophotocoagulation, goniotomy, goniosynechialysis and internal revision of glaucoma filtration operations. Furthermore in addition to diagnosis and treatment of glaucoma, gonioscopy is often used in the diagnosis and management of ocular trauma, intraocular foreign bodies, and complications of intraocular surgery.

Gonioscopy uses a goniolens which is an optical device that is used to capture the incident angle of light reflected from the anterior chamber angle which is greater than the critical angle at the cornea-air interface.

A common lens utilized in connection with gonioscopy is known as the Swan-Jacob Gonioprism (the “Swan”) lens. The Swan lens comprises a contact lens having a posterior contact surface that conforms to the anterior surface of the cornea of an eye. The contact surface is generally spherical and has an optical axis that may be aligned with the optical axis of the eye. The contact lens also has an anterior surface that is offset in an anterior direction from the contact surface.

When the contact lens is positioned on the eye that is coated with a lubricating coupling fluid, the user may view the anterior chamber by looking into the anterior surface in a direction generally parallel to the optical axis of the anterior surface of the lens. The contact surface is typically smaller than the cornea so that the lens can be moved around on the cornea to view various parts of the anterior chamber.

Traditional use of this type of lens requires the user to hold and stabilize the lens on the surface of the cornea for the duration of the procedure. This leaves the physician with only one free hand to perform all maneuvers required during surgery. The hand held technique can be acceptable for diagnostic procedures but the use of a dedicated hand used for this purpose during surgical intervention is not ideal. The availability of a second hand is a great asset as it can be used to control additional instrumentation or to manipulate the eye during the procedure. Unanticipated surgical complications frequently occur and the availability of a second hand to assist in the management of problems can make the difference between surgical success and failure. It is this need that can be addressed with the Suspended Goniolens System.

### SUMMARY OF THE DISCLOSURE

Optical systems are disclosed herein. The optical systems include a balance arm having a first end, a second end, and a pivot point positioned between the first and second ends; a movable counterbalance weight disposed on the balance arm between the pivot point and the second end; and a goniolens disposed on the balance arm towards the first end of the balance arm. The goniolens can be configured to contact and apply an adjustable force to a patient's eye based on a position of the movable counterbalance. The goniolens can be configured to rotate relative to the balance arm. The movable counterbalance weight can be slidable along the balance arm. Sliding the movable counterbalance weight towards the second end controls a force applied to the eye with the goniolens.

The optical systems can include a lateral control mechanism configured to control the lateral position of the goniolens. The optical systems can include a vertical control mechanism configured to control the vertical position of the goniolens. The optical systems can include a rotational control mechanism configured to rotate the balance arm and goniolens.

The optical systems can include an adapter configured to connect the balance arm to a microscope. The goniolens can be configured to move or swivel from a first position in line with an optical path of the microscope to a second position out of the line of the optical path of the microscope.

Methods for using a gonioscope are provided herein. The methods can include positioning a microscope over a patient's eye so as to put the eye in an optical path of the microscope; positioning a goniolens in an area adjacent to a patient's eye, the goniolens being attached at one end of a balance arm; and adjusting a counterweight disposed on the balance arm to contact the goniolens with the patient's eye with a desired contact pressure. The methods can include performing a medical procedure on the patient's eye using two hands. The medical procedure can include a surgical procedure such as implantation of a drainage device, laser trabeculoplasty, peripheral laser gonioplasty, goniophotocoagulation, goniotomy, goniosynechialysis, or internal revision of glaucoma filtration operations.

Optical systems are also provided including a surgical microscope defining an optical plane; a balance arm connected to the surgical microscope, the balance arm having a first end, a second end, and a pivot point positioned between the first and second ends; a movable counterbalance weight disposed on the balance arm between the pivot point and the second end; and a goniolens disposed on the balance arm towards the first end of the balance arm. The goniolens can be configured to contact and apply an adjustable force to a patient's eye in the optical plane based on a position of the movable counterbalance. The counterbalance can include a first weight disposed on the balance arm and a second weight disposed about the first weight. The first weight can be movable relative to the balance arm and the second weight can be movable relative to the first weight. The position of the second weight can be moved to apply a known force on the eye.

### BRIEF DESCRIPTION OF THE FIGURES

FIG. 1 is a stylized of a view of a hand held Swan-Jacob gonioprism positioned on a surface of an eye.

FIG. 2 is a stylized of a view of a gonioscopic surgery showing the utility of a surgeon's hands.

FIG. 3 is a front perspective view of the suspended goniolens system in accordance with an embodiment.

FIG. 4 is a rear perspective view of the suspended goniolens system in accordance with an embodiment.

FIG. 5A is a perspective view of the microscope head and the adapter used to attach a suspended goniolens system in accordance with an embodiment.

FIG. 5B is another perspective view of the microscope head and the adapter used to attach the suspended goniolens system in accordance with an embodiment.

FIG. 6 is a front perspective view of the suspended goniolens system in a lowered position in accordance with an embodiment.

FIG. 7A is a side view of the balance arm of the suspended goniolens system in accordance with an embodiment.

FIG. 7B is a cross-sectional view of the balance arm of the suspended goniolens system in accordance with an embodiment.

FIG. 7C is an enlarged cross-sectional view of the counterbalance weight of the balance arm of the suspended goniolens system in accordance with an embodiment.

FIG. 8A is an enlarged view of the goniolens and balance arm in accordance with an embodiment.

FIG. 8B is an enlarged view of the goniolens and balance arm in accordance with an embodiment.

FIG. 8C is an enlarged view of the goniolens and balance arm in accordance with an embodiment.

FIG. 9A is an enlarged detailed cross-sectional view of the suspended goniolens system in the extended position in accordance with an embodiment.

FIG. 9B and 9C are enlarged cross-sectional views of the slide arm of the suspended goniolens system in accordance with an embodiment.

FIG. 10A is a perspective view of the microscope head and adapter with the suspended goniolens elevated above the eye in accordance with an embodiment.

FIG. 10B is a perspective view of the microscope head and adapter with the suspended goniolens contacting the eye in accordance with an embodiment.

FIG. 11A is a perspective view of the goniolens contacting the eye at a first location in accordance with an embodiment.

FIG. 11B is a perspective view of the goniolens contacting the eye at a second location in accordance with an embodiment.

FIG. 12 is a perspective view of the microscope with the adapter and the suspended goniolens system rotated away from the optical axis in accordance with an embodiment.

FIG. 13 is a perspective view of the microscope with the entire adapter and the suspended goniolens system rotated away from the optical axis in accordance with an embodiment.

### DETAILED DESCRIPTION

Suspended goniolens systems and devices are disclosed herein. Methods for using the suspended goniolens systems and devices are also disclosed. The suspended goniolens system can be configured to hold and position a goniolens in contact with a patient's eye without requiring a hand or other input to hold the goniolens in the desired position. In contrast, a traditional handheld goniolens has to be held in place during the procedure, preventing the clinician from using both hands during the procedure.

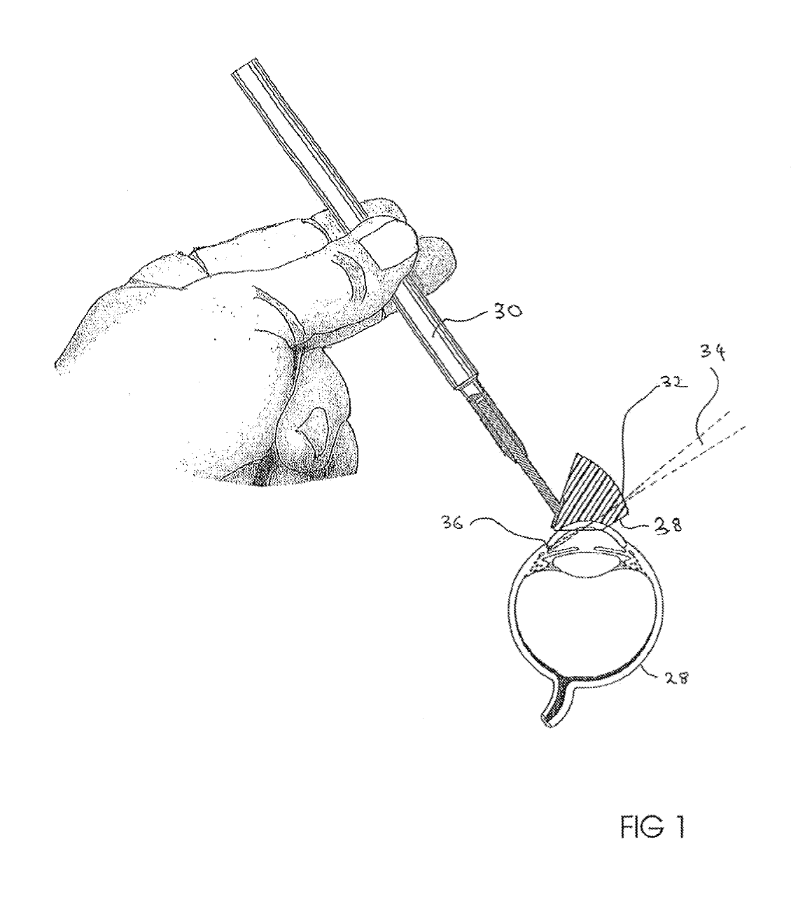

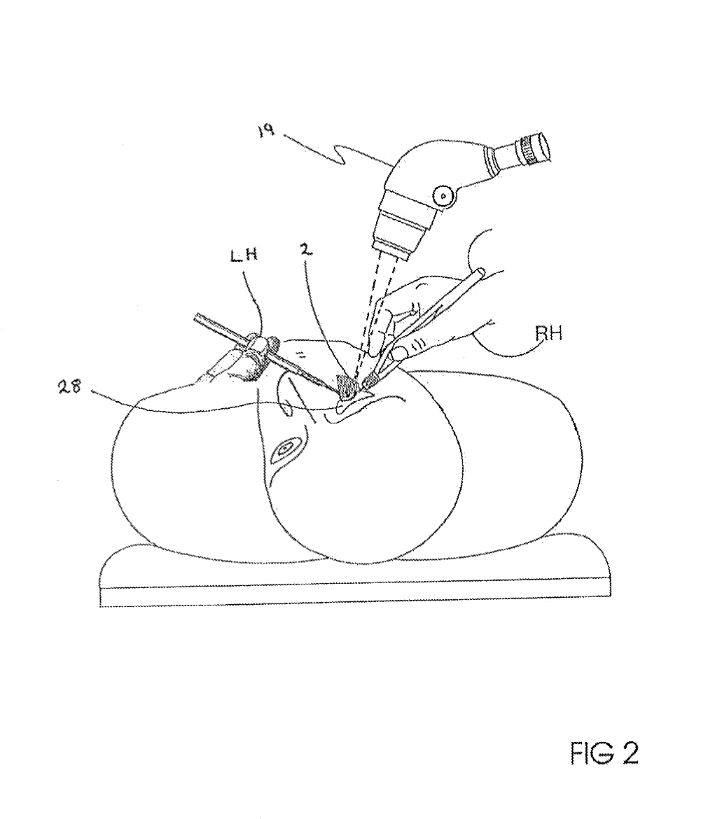

FIGS. 1 and 2 illustrate procedures utilizing a traditional gonioscope. FIG. 1 is stylized representation of a gonioscopy procedure in which a physician is holding a hand piece 30 of a Swan-Jacob Gonioprism 38 in his or her hand in order to view the anterior chamber angle of an eye 28. The focal point 32 of the gonioprism 38 is aligned with the viewing focus 34 of a microscope and the anatomical feature of interest 36. The use of the gonioprism requires considerable skill and care. If excessive pressure is applied to the surface of the eye, the angle of the eye can be altered and a misdiagnosis made. In addition unintentional rotation of the lens can cause striations on the cornea which will create a distortion in the viewed image.

FIG. 2 is stylized representation of a surgical procedure in which a surgeon is utilizing gonioscopy during the implantation of a glaucoma drainage device. The left hand is used to hold and stabilize the gonioscope while the right hand simultaneously injects the glaucoma drainage device via an appropriate delivery system. Visual observation of the procedure is necessary and occurs concurrently via microscope 19. It can be recognized that using a dedicated hand to hold a gonioscope in this manner means that the implant procedure must be performed as a single handed operation. During surgical procedures of this nature unanticipated complications can occur and an additional hand would allow the surgeon to better manage the situation.

The suspended goniolens systems disclosed herein allow for the clinician to use both hands during a procedure. The ability to use both hands offers many advantages. The additional hand can be used to hold another instrument, stabilize an external surface of the eye, and treat and prevent any complications that may arise during the procedure. For example, another instrument can be used to restrict eye movement of a non-compliant patient, manipulate the cornea to encourage optimal delivery of an optical implant, irrigate and aspirate refluxed blood from the visual field during the delivery of an implant, and perform other useful steps during the procedure. The ability to use both hands can also reduce the stress on the clinician because of the knowledge that the free hand can be used if complications occur.

The suspended goniolens systems of this disclosure can include a balance arm with a goniolens disposed on one end and a counterbalance weight disposed on the other end of the balance arm. The weight can be adjusted to control the position of the goniolens. The weight can also be adjusted to control the pressure applied to the patient's eye by the goniolens. As discussed in greater detail below, the weight can be designed such that the goniolens provides a precise contact force on the patient's eye.

The design of the balance arm and the counterbalance weight can also prevent the goniolens from applying too large of a force on the patient's eye. The maximum force that can be applied to the eye by the goniolens can be determined based on the controlled counterbalance weight. The suspended goniolens systems disclosed herein can provide improved safety to the patient.

The goniolens can include a concave surface to facilitate engagement with the cornea 100 of the patient's eye. A coupling fluid, such as a viscoelastic fluid, can be applied to the surface of the cornea. The viscoelastic fluid can facilitate the contact between the goniolens and the eye and improve the optical view of the eye. Air bubbles can form in the viscoelastic fluid. Some pressure can be applied by the goniolens such that the air bubbles are pushed away from the interface and are moved out of the contact area between the goniolens and the eye. If a larger force is applied by the goniolens then the surface of the eye can be deformed by the downward force of the goniolens. The deformation can adversely affect the visualization of the anatomical angle formed between the eye's cornea and iris (the irideocorneal angle) and result in a misdiagnosis. It is desirable to contact the goniolens with the eye such that the normal surface geometry of the cornea is maintained.

The goniolens can engage with the balance arm such that the goniolens can rotate relative to the balance arm. The free rotation of the goniolens can improve the concentric alignment of the goniolens with the patient's cornea 100. The rotation of the goniolens can also improve concentric alignment with the patient's cornea when the goniolens moves across the patient's eye.

The suspended goniolens systems disclosed herein can be used with microscopes. Examples of microscopes include optical microscopes and surgical microscopes. The suspended goniolens systems can be attached to an adapter that engages with the microscope. In some embodiments the suspended goniolens system can be attached to the microscope. The suspended goniolens system can be configured to align the goniolens in focus with the optical axis of the microscope prior to the patient being on the operating table. The microscope can include a control system to move the head of the microscope. The suspended goniolens system can be coupled to the head of the microscope such that the suspended goniolens system moves with the microscope head and the goniolens stays in focus. The microscope control system can include foot controls for adjusting the position of the microscope. The foot controls can be operated by the clinician during a medical procedure without requiring the use of the clinician's hands.

The suspended goniolens system can be used for gonioscopy or various medical procedures that use a goniolens. The medical procedure can include surgical procedures. Examples of surgical procedures include implantation of a drainage device, laser trabeculoplasty, peripheral laser gonioplasty, goniophotocoagulation, goniotomy, goniosynechialysis, or internal revision of glaucoma filtration operations.

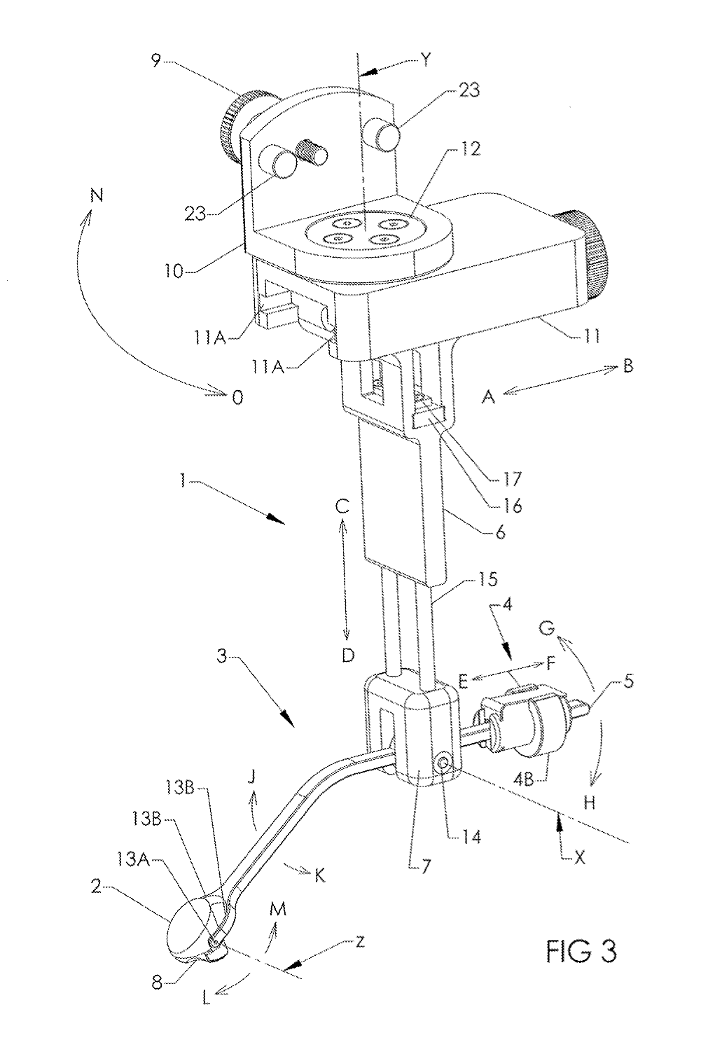

The suspended goniolens system can allow for positioning the goniolens with a number of degrees of freedom, as illustrated in the Figures and discussed in detail below. For example, the goniolens system can allow for lateral movement of the goniolens, vertical movement of the goniolens, and rotational movement. The lateral movement of the goniolens can be controlled by a lateral control mechanism. The lateral control mechanism can include a screw mechanism that can be turned to move the goniolens laterally, such as along axis A-B illustrated in FIG. 3. The vertical movement of the goniolens can be between a raised and lowered position.

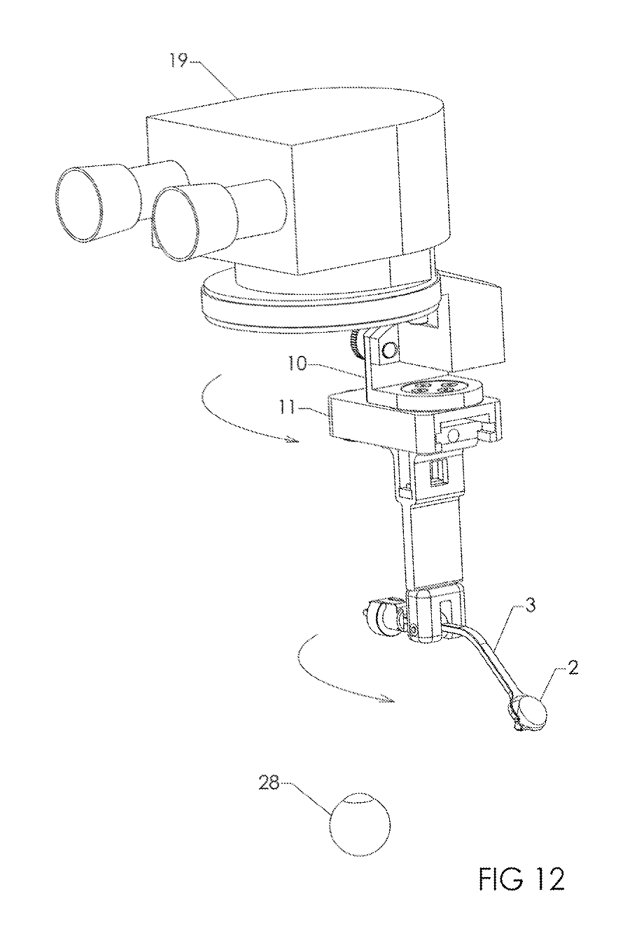

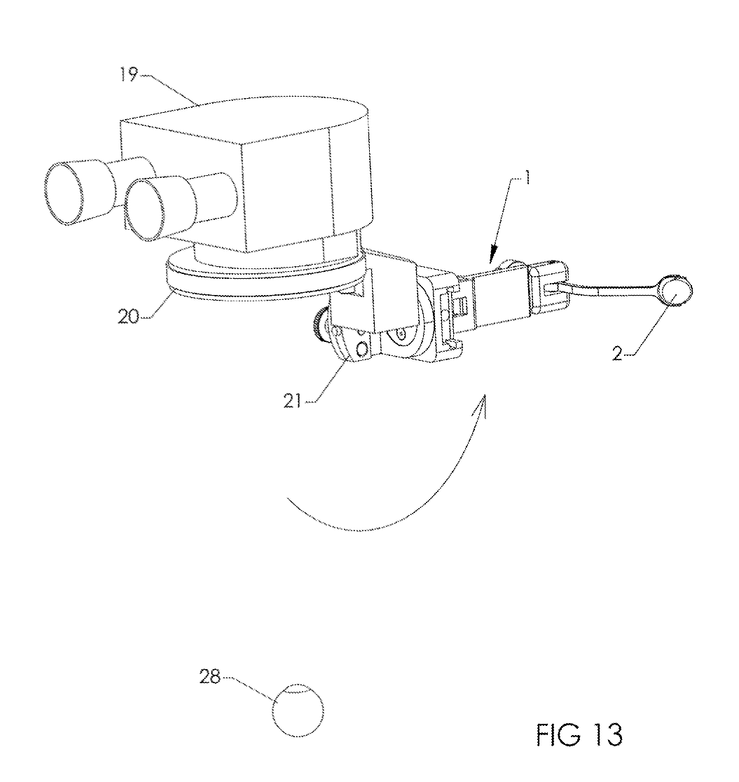

The rotational movement can include rotation about one or more planes or axes. Rotation about one axis can rotate the goniolens away from the optical axis (as shown in FIG. 12). Rotation about a second axis can rotate the entire goniolens system (as shown in FIG. 13). The goniolens can be rotated back into the optical axis without requiring refocusing or recalibrating the positioning of the goniolens.

The suspended goniolens system can be made out of surgical grade materials that can withstand sterilization temperatures. In some embodiments the suspended goniolens system is sterilized in an autoclave between surgical procedures. Examples of suitable metals include stainless steel, coated aluminum, titanium, and other metals that can withstand sterilization temperatures. Suitable non-metal materials include polymers and plastics that can withstand sterilization temperatures. Examples of non-metals include silicone, polyetheretherketone (PEEK), polyetherimides (PEI or ULTEM®), and (Teflon) Polytetrafluorethylene.

The following detailed description should be read with reference to the drawings in which similar elements in different drawings are numbered the same. The drawings, which are not necessarily to scale, depict illustrative embodiments and are not intended to limit the scope of the invention.

FIG. 3 is a front perspective view of a Suspended Goniolens System 1 in accordance with one embodiment. The goniolens system 1 includes a goniolens 2. The goniolens 2 can include a concave surface 8 shaped to match the surface of the cornea 100. The suspending goniolens system 1 includes features which enable lateral, rotational, and vertical movement and positioning of the goniolens. The goniolens 2 can be attached to one end of a balance arm 3 which pivots about pivot point 14. Pins 13A on the goniolens can engage holes on the balance arm to allow independent rotation of the gonio lens with respect to the balance arm. The balance arm 3 can include flexible arms 13B that can straddle the goniolens 2 and hold it in place. The flexible arms 13B can also allow the goniolens 2 to be readily removed for cleaning and/or replacement. Attached on the opposite end 5 of the balance arm is a weight 4 configured to counterbalance the goniolens about the pivot point 14. The weight 4 can be slidably disposed on the balance arm, for example. The balance arm 3 and pivot point 14 can be engaged with the bottom block 7. In one embodiment, bottom block 7 is attached to one end of the slide rods 15 with end plate 17 attached to the opposing ends of slide rods 15. Top slide block 6 includes a stop plate 16. Top slide block 6 is configured to receive the slide rods 15 and end plate 17 such that the slide rods 15 and end plate 16 are movable vertically relative to the top slide block 6. The top slide block 6 is configured to engage with the grooves 11A of the top slide 11. The top slide block 6 can move laterally along the grooves 11A of the top slide 11. The lateral movement of top slide block 6 can be controlled using a lateral control mechanism 18. The top slide 11 can engage with a pivot bracket 10. The pivot bracket 10 includes a plate 12 and pins 23. The plate 12 can engage with the top slide 11 to allow rotation of the top slide 11 relative to the pivot bracket 10. The suspended goniolens system can be attached to an adapter engaged with a microscope using pins 23 and screw 9.

The suspended goniolens system 1 in FIG. 3 illustrates a number of adjustable features, including lateral alignment along axis A-B (via slide block 6 moving laterally along grooves 11A), vertical alignment along axis C-D (via slide rods 15), counterbalance weight movement along E-F, balance arm movement along G-H and J-K, goniolens rotation along L-M, and rotation between the top slide 11 and pivot bracket 10 about axis X in directions N-O. The adjustable features allow for improved positioning of the goniolens.

The lateral alignment of the goniolens can be adjusted by moving the top slide block 11 along axis A-B. The lateral control mechanism 18, illustrated as a thumb screw, can be used to move the top slide block 6. For example, the thumb screw can engage a thread and can rotate to move the top slide block 6 relative to top slide 11 along axis A-B. The lateral alignment can be adjusted along axis A-B to position the goniolens on the optical axis of the microscope.

The vertical position of the goniolens can be adjusted along axis C-D by sliding the slide rods 15 and lower block 7 relative to the top slide block 6. The lower block 7 and the balance arm assembly can move between an elevated position and a lowered position. When the lower block 7 and balance arm 3 are in a lowered position the end plate 17 can contact the stop plate 16 of the slide block 6. When the lower block 7 and balance arm 3 are in an elevated position the lower block 7 can contact the bottom of slide block 6. The slide rods 15 and slide block 6 can be configured to hold the position of the slide rods 15 in place at any position between the elevated and lowered positions.

The goniolens 2 can be engaged with the balance arm 3 such that it can freely rotate. For example, referring to FIG. 3, the goniolens 2 can be connected to the flexible arms 13B of the balance arm 3 with pins 13A on opposing sides of the goniolens 2. The goniolens 2 can rotate about axis Z in directions L-M. The rotation of the goniolens can facilitate concentric alignment with the patient's cornea. The rotation also can allow for the goniolens to maintain concentric alignment with the patient's cornea while moving the goniolens across the patient's eye.

The balance arm 3 can rotate about pivot axis X created by the pivot point 14. The balance arm 3 can rotate in directions J-K and G-H with the position controlled by gravitational forces. The pivot alignment can be adjusted by moving counterbalance weight 4 along the balance arm 3 in direction E-F. Moving the counterbalance weight 4 along the balance arm 3 in direction E-F changes the force applied by the counterbalance weight 4 thereby causing the balance arm 3 to pivot about the pivot axis X. The balance arm can rotate in direction G-H on the counterbalance weight side of the balance arm and correspondingly in direction J-K on the goniolens side.

The goniolens and balance arm can also rotate relative to the pivot bracket about axis Y in directions N-O. The top slide 11 can rotate relative to pivot bracket 10 thereby rotating the goniolens 2 and the balance arm 3. The top slide 11 can rotate between multiple discrete positions, such as between 90° angles. In some embodiments the top slide 11 can rotate to any point along the 360° axis. In some embodiments, the rotation mechanism can include a spring loaded ball (not shown) located in top slide 11. The spring loaded ball can engage a complimentary recessed hole in the pivot bracket 10 (not shown) in the aligned position so that the aligned relationship can be quickly reestablished when the goniolens is returned to the aligned position.

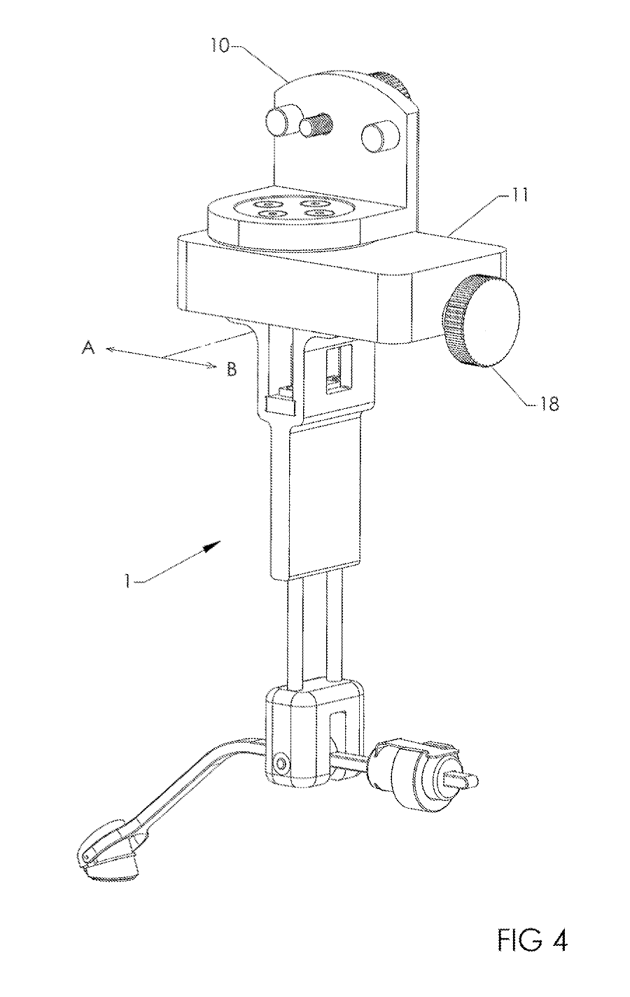

FIG. 4 illustrates a rear perspective view of the suspended goniolens system 1 shown in FIG. 3. Lateral control 18, illustrated as a thumb screw, can engage a complimentary thread in top slide block 6. The lateral alignment can be used to fine tune the positioning of the goniolens 2 relative to the optical axis and focus of the microscope.

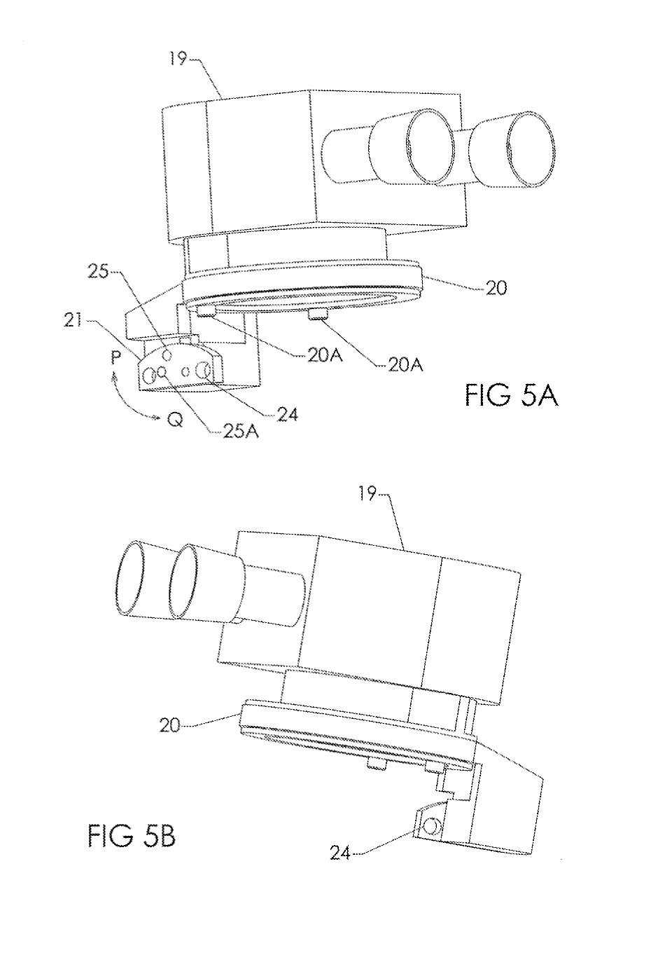

FIGS. 5A & 5B show perspective views of a microscope head and an adapter used to attach the suspended goniolens system to the microscope head. In some embodiments the suspended goniolens system does not directly attach to the microscope head or objective. An adapter 20 can be attached to the microscope 19 with thumb screws 20A. The adapter 20 can include a mounting plate 21 with pin holes 24, a threaded hole 25 and a spring loaded pivot pin 25A. The suspended goniolens system can be attached to the adapter 20 using the screw 9 and pins 23 illustrated in FIG. 3. The screw 9 can engage with the threaded hole 25. The pins 23 can engage with the pin holes 24. The mounting plate 21 can be configured to rotate relative to the other parts of the adapter 20 via an axis created by pin 25A. The mounting plate 21 can be configured to rotate in directions P-Q. The suspended goniolens system can rotate with the mounting plate thereby rotating in relation to the microscope and optical axis. The mounting plate 21 can have a two position spring controlled mechanism which has sufficient force to hold the suspended gonio system in either the raised or lowered position.

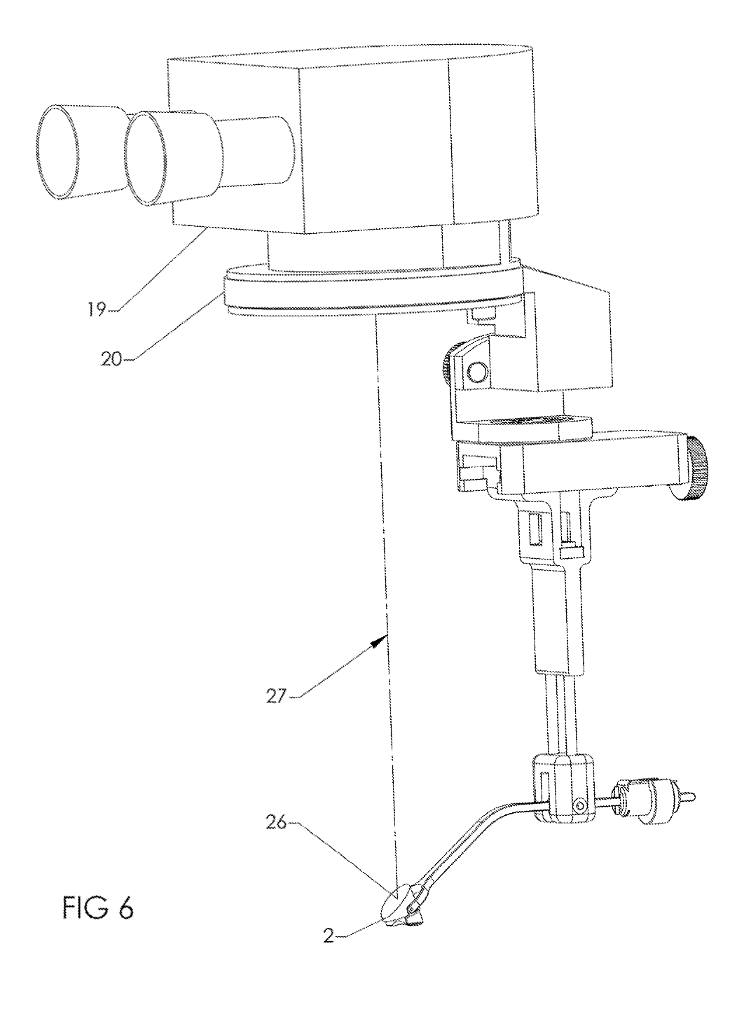

FIG. 6 is a front perspective view of the suspended goniolens system 1 attached to the adapter 20 with the goniolens 2 in a lowered position. The adapter 20 is engaged with the microscope 19. As shown, the goniolens 2 is aligned in the optical axis or optical path 27 of the microscope 19. The adjustable features of the suspended goniolens system can be used to position and focus the goniolens 2 relative to the optical axis 27 and focal point 26 of the surgical microscope 19, either prior to or after the patient's arrival on the operating table. The suspended goniolens system can be adjusted to weightlessly suspend the goniolens 2 in the optically focused position with respect to the optical axis 27 of the microscope 19. The suspended goniolens system is ready to be used by a clinician after the goniolens 2 is in an optically focused position.

FIG. 6 illustrates the microscope in a vertical arrangement with the suspended goniolens system. The skilled artisan will appreciate that the microscope can also be used tilted at an angle. For many procedures the microscope is titled at an angle using the tilt feature that is a common feature on microscopes in order to improve the view of the patient's eye, as shown in FIG. 2. The patient's head can also be tilted to improve the view of the patient's eye, as shown in FIG. 2. The goniolens 2 and balance arm 3 can be calibrated and positioned based on the desired orientation of the microscope to the patient. If the angle of the microscope is changed then the counterweight 4 can be re-positioned and the top side adjusted to re align the goniolens 2 with the optical axis 27 of the microscope 19.

After alignment, the goniolens 2 can be raised to an elevated position and rotated out of the optical focus. The goniolens 2 can be lowered and rotated back in to the optically focused position when the patient is ready for the procedure. With the suspended goniolens system the clinician is free to use the hand that is normally dedicated to holding the gonioprism during the gonioscopy procedure illustrated FIG. 2.

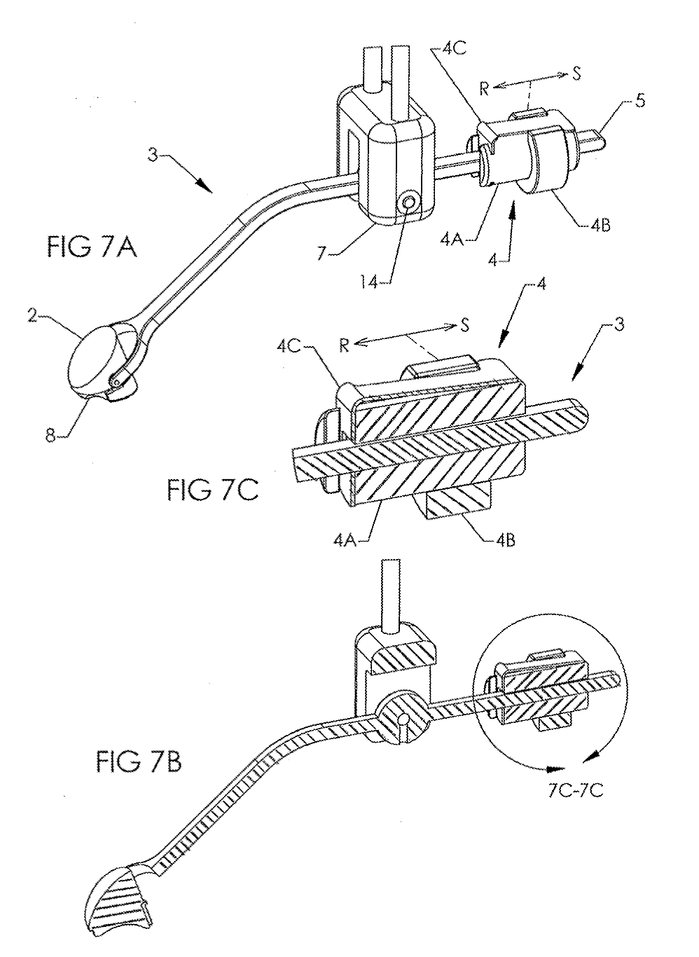

FIG. 7A-7C illustrate various views of the balance arm 3. FIG. 7A shows the entire balance arm and associated components, FIG. 7B shows a cross-sectional view of the balance arm, and FIG. 7C is a zoomed view of the cross-sectional counterweight 4 from circle 7C-7C of FIG. 7B. The counterweight 4 can include a first weight 4A, second weight 4B, and locking spring 4C. The first weight 4A is disposed about the balance arm 3. The second weight 4B is illustrated as engaging with an exterior of the first weight 4A. The second weight 4B can be referred to as a cornea coupling weight. The second weight 4B is slidable with respect to the first weight 4A in direction R and direction S. The locking spring 4C can be used to hold the first weight 4A in a desired position on the balance arm. The locking spring 4C can be bent at an angle to hold the counterweight 4 in place on the balance arm 3 by applying pressure to the counterweight 4 and balance arm 3. For example, the locking spring 4C can engage the balance arm 3 and first weight 4A to prevent movement of the first weight 4A relative to the balance arm 3. The locking spring 4C can be moved to a non-engaged position, such as by pushing down the locking spring 4C, to allow for movement of the counterweight 4 during calibration of the goniolens 2. Pushing down on the locking spring 4C can disengage the locking spring 4C from contacting the balance arm 3, thereby allowing for movement of the counterweight 4.

Referring to FIGS. 6 and 7A-7C, calibration and positioning of the goniolens in the optical axis 27 and focal point 26 of the microscope 19 will now be discussed. In one embodiment, the second weight 4B can be moved in direction S further away from the goniolens to reduce the downward force applied by the goniolens or move the balanced position of the goniolens. After weightlessly positioning the goniolens in the focal point 26, the goniolens is ready to be placed into contact with the patient's eye. At the beginning of the procedure the goniolens can be positioned into contact with the patient's eye and any viscoelastic fluid on the surface of the eye. The goniolens initially can provide very little contact pressure to the eye because the goniolens was positioned to be balanced weightlessly in the focal point 26 of the microscope. Next, a desired contact pressure can be applied from the goniolens to the eye by sliding the second weight 4B in the R direction towards the goniolens 2. Moving the second weight 4B decreases the force from the counterweight and increases the downward force on the patient's eye from the goniolens 2. The second weight 4B can be sized to provide the desired contact pressure on the patient's eye. It is desirable to apply enough pressure to the viscoelastic fluid to dispel bubbles; however, if too much pressure is applied the irideocorneal angle of the eye will be squeezed and the viewing of interior structures of the eye will be compromised. The mass of the second weight 4B can be selected to achieve the desired goniolens contact pressure on the eye. The pressure on the eye can be precisely determined based on the mass of the counterweight 4, mass of the second weight 4B, and distance that the second weight 4B moves along the balance arm axis. The balance arm 3 design can also provide a safeguard against contacting the goniolens 2 to the patient's eye with too much force because of the free movement of the goniolens 2.

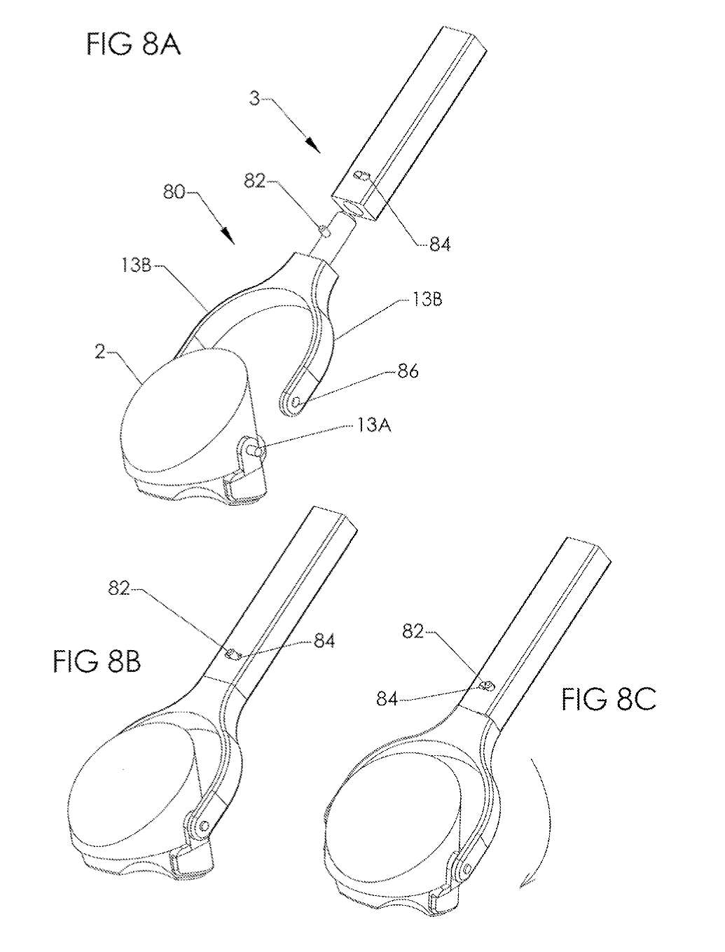

FIG. 8A is an enlarged view of the goniolens and balance arm in accordance with an embodiment. FIG. 8A shows the separate pieces of the goniolens side of the balance arm, including the goniolens 2, goniolens holder 80, and balance arm 3. The balance arm 3 includes a balance arm slot 84 adapted to engage with a pin 82 on the goniolens holder 80. The goniolens holder 80 includes the flexible arms 13B and holes 86 for engaging the goniolens pins 13A. FIG. 8B is a view of the sections illustrated in FIG. 8A in an assembled configuration. The pin 82 can move within the balance arm slot 84 to rotate the goniolens holder 80 and goniolens 2. FIG. 8B illustrates the goniolens holder 80 with the pin 82 aligned in a central position with the balance arm slot 84. FIG. 8C illustrates the goniolens holder 80 in a rotated position with the pin 82 touching a side of the balance arm slot 84. The additional degree of freedom for rotating the goniolens holder 80 can further improve the goniolens 2 engagement with the patient's eye.

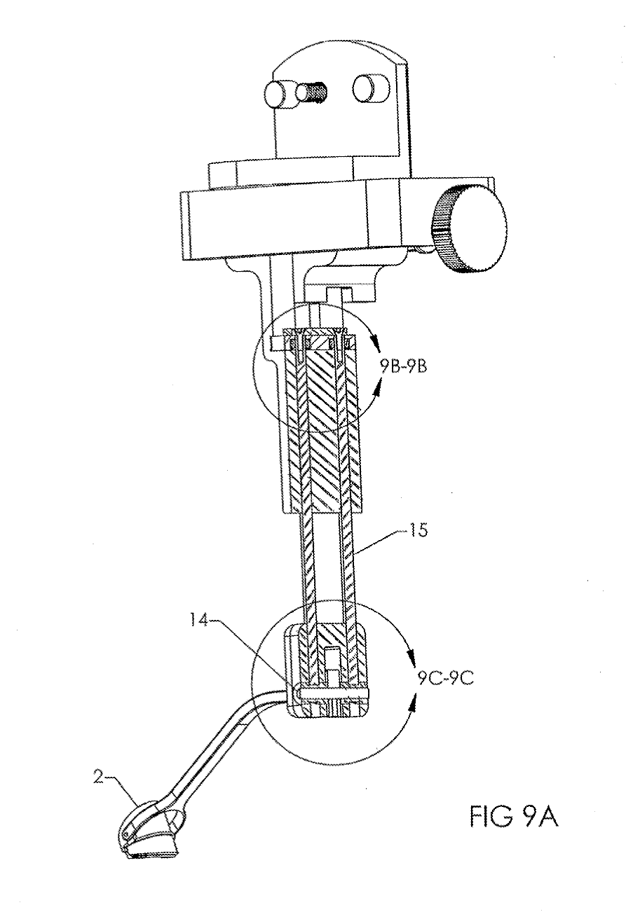

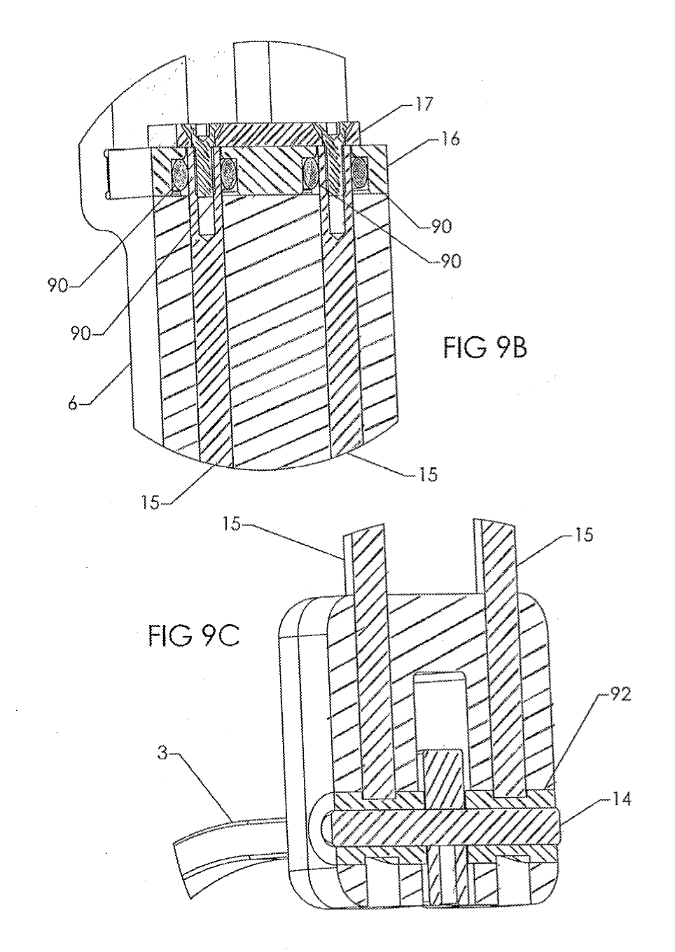

FIG. 9A is a perspective view of a portion of the suspended goniolens system with the top slide block 6 sectioned to show the features of the internal components. FIGS. 9B and 9C illustrate enlarged portions of FIG. 9A along sections 9B-9B and 9C-9C, respectively. FIG. 9B shows o-rings 90 disposed about the slide rods 15 and within the stop plate 16. The o-rings 90 provide compression and friction to the slide rods 15 to hold the slide rods 15 in any elevated position along with the lower block 7 and balance arm 3. FIG. 9C illustrates a cross-section of the lower block 7. The slide rods 15 are illustrated along with a bearing 92 disposed about the pivot point 14.

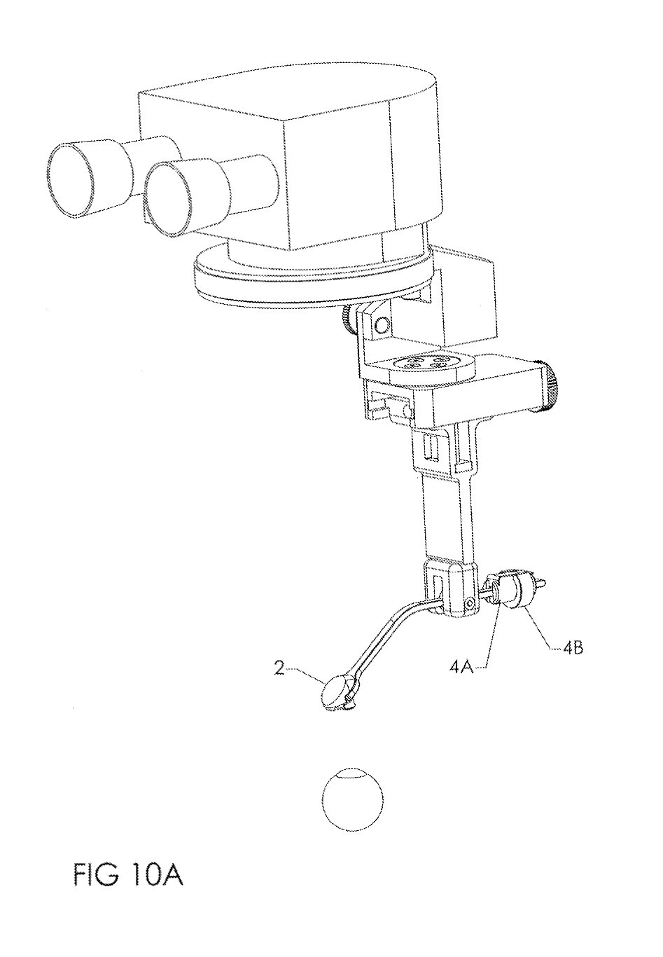

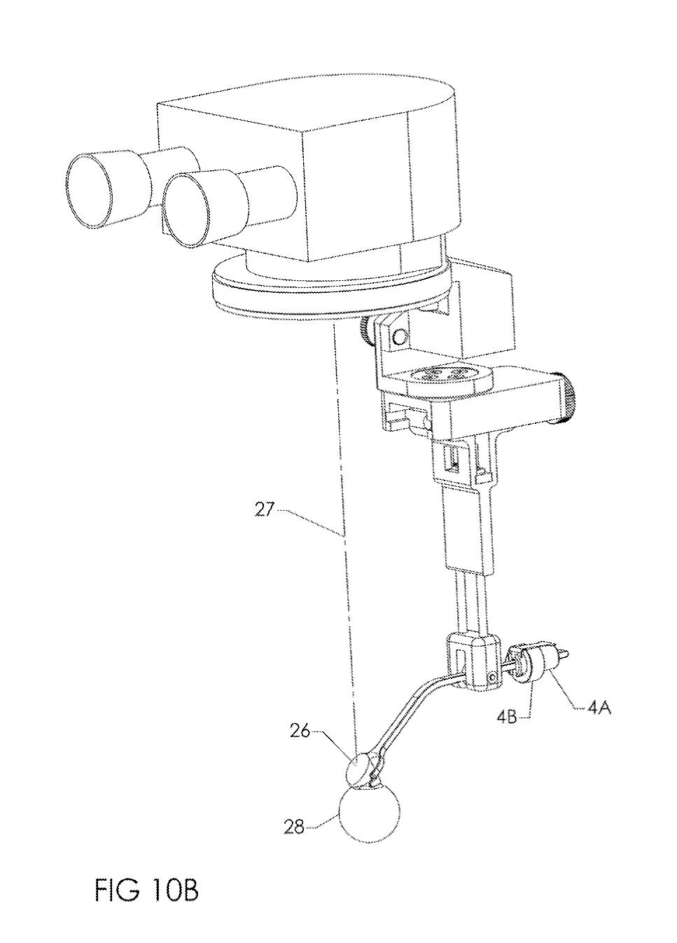

FIG. 10A is a front perspective view of the suspended goniolens system 1 attached to the surgical microscope 19 in an elevated position with the goniolens 2 elevated from the eye 28. Bottom block 7 is illustrated pushed upward and in contact with the top slide block 6. The second weight 4B is positioned away from the goniolens 2 in FIG. 10A. The suspended goniolens system is configured in an elevated position prior to placing the device on the patient's eye 28 or after the procedure is finished. When the suspended goniolens system is in an elevated position, additional clearance is provided between the goniolens and the eye. The additional clearance can allow the clinician to safely maneuver and position the device with respect to the patient.

FIG. 10B is a perspective view of the microscope head 19 and adapter 20 with the goniolens 2 contacting the eye 28. The goniolens 2 is lowered into contact with the eye 28 and the second weight 4B is moved towards the goniolens 2 to apply a controlled coupling force to the eye by the goniolens 2. The goniolens 2 is positioned in the focal point 26 and optical axis 27. The suspended goniolens system configuration illustrated in FIG. 10B is in position and ready for a surgical procedure.

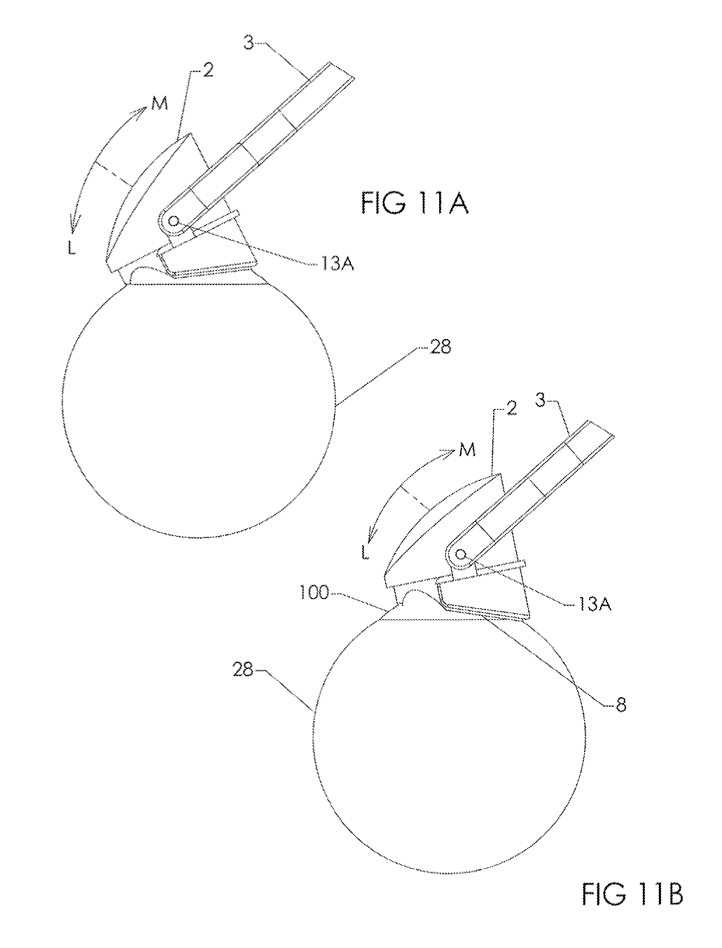

FIG. 11A is a perspective view of the goniolens contacting the eye at a first location. FIG. 11B is a perspective view of the goniolens contacting the eye at a second location. The goniolens 2 can be precisely repositioned on the patient's eye 28 as necessary to optimize the view of the irideocorneal angle. Surgical microscopes are typically equipped with integrated assistant functions that are foot operated. These allow the physician to precisely manipulate the head 19 which in turn moves the entire suspended goniolens assembly 1. In some embodiments the vertical, horizontal, and rotational alignment of the suspended goniolens system can be used to adjust the position of the goniolens on the eye. The posterior surface of the goniolens 2 has a radius which is substantially the same as that of the cornea 100. It will be appreciated that as the goniolens 2 is moved over the surface of the eye the it can freely rotate about pin 13 and thus maintain a concentric relationship with the cornea 100 as it moves across the eye 28.

FIG. 12 is a perspective view of the microscope head 19 and adapter 20 with the suspended goniolens system 1 rotated away from the optical axis of the microscope. The top slide 11 is rotated 90° relative to the pivot bracket 10. The rotation of the top slide 11 also rotates the goniolens 2 and balance arm 3 relative to the pivot bracket 10 and microscope 19. The goniolens 2 can be rotated away from the optical axis of the microscope and rotated back into the optical axis without the need to re-focus or reposition the goniolens 2. During a medical procedure the clinician can want a direct image of the eye without the goniolens 2. The goniolens 2 can be rotated out of the optical axis to allow for a direct image of the eye. When the goniolens 2 is needed the goniolens 2 is rotated back into the optical axis. The suspended goniolens system can also be rotated away from the optical axis during surgery to provide additional access space for the clinician, if needed. The suspended goniolens system can also be rotated away from the optical axis and surgical site after the completion of surgery.

FIG. 13 is a perspective view of the microscope 19 with the entire adapter and the suspended goniolens system 1 rotated away from the optical axis. The suspended goniolens system is rotated away from the optical axis of the microscope in a different direction than the direction shown in FIG. 12. The mounting plate 21 of the adapter 20 is rotated 90° along axis P-Q illustrated in FIG. 5A along with the entire suspended goniolens system 1. The spring biased mounting plate 21 can be rotated between positions using spring biasing.

### EXAMPLE 1

A suspended goniolens system was sterilized in an autoclave. The sterilized suspended goniolens system was then attached to an optical surgical microscope. The suspended goniolens was attached to an adapter attached to the head of the optical surgical microscope. The orientation of the optical axis of the microscope was set based on the procedure to be performed on the patient. The suspended goniolens system was calibrated in a lowered configuration to position the goniolens in the optical axis of the microscope. The counterbalance weight was adjusted along the balance arm to weightlessly balance the goniolens in the optical focus of the optical surgical microscope.

After the goniolens was calibrated in the focused position the suspended goniolens was moved to a raised position and rotated away from the optical axis. The suspended goniolens was then ready for the patient.

### EXAMPLE 2

The patient is positioned on the operating table in the desired orientation to the optical axis of the optical surgical microscope. A viscoelastic fluid is applied to the surface of the eye that is to be treated by the procedure. After the patient is in the desired position the suspended goniolens system is rotated to align it with the optical axis. The goniolens is then lowered into contact with the patient's eye. The cornea coupling weight is then slid towards the goniolens to apply a desired contact force to the patient's cornea by the goniolens. The medical procedure then begins. The clinician can use both hands during the medical procedure because the goniolens is positioned in place by the suspended goniolens system. The position of the goniolens can be moved along the patient's eye as needed. The position of the microscope head is moved using the microscope positioning controls, such as foot controls. The suspended goniolens system moves with the microscope head. The goniolens can rotate to maintain contact with the patient's eye and stays in the microscope focus.

After the procedure is done the goniolens is raised to an elevated position and rotated away from the optical axis. The suspended goniolens system is then removed from the microscope and sterilized in an autoclave.

The foregoing detailed description of the technology herein has been presented for purposes of illustration and description. It is not intended to be exhaustive or to limit the technology to the precise form disclosed. Many modifications and variations are possible in light of the above teaching. The described embodiments were chosen in order to best explain the principles of the technology and its practical application to thereby enable others skilled in the art to best utilize the technology in various embodiments and with various modifications as are suited to the particular use contemplated. The present invention descriptions are intended to cover such alternatives, modifications, and equivalents as may be included within the spirit and scope of the invention as defined by the appended claims and otherwise appreciated by one of ordinary skill in the art.

What is claimed is:

1. An optical system, comprising:
a balance arm having a first end, a second end, and a pivot point positioned between the first and second ends;
a movable counterbalance weight disposed on the balance arm between the pivot point and the second end; and
a goniolens disposed on the balance arm towards the first end of the balance arm, the goniolens configured to contact and apply an adjustable force to a patient's eye based on a position of the movable counterbalance.

2. The system of claim 1, wherein the goniolens is configured to rotate relative to the balance arm.

3. The system of claim 2, wherein the goniolens is configured to self-align to a cornea of the patient's eye.

4. The system of claim 1, further comprising an adapter configured to connect the balance arm to a microscope, wherein the goniolens is configured to swivel from a first position in line with an optical path of the microscope to a second position out of the line of the optical path of the microscope.

5. The system of claim 4, wherein the microscope comprises a surgical optical microscope.

6. The system of claim 1, wherein the optical system does not attach to an objective of a microscope.

7. The system of claim 1, wherein the movable counterbalance weight is slidable along the balance arm.

8. The system of claim 7, wherein sliding the movable counterbalance weight towards the second end controls a force applied to the eye with the goniolens.

9. The system of claim 1, wherein the movable counterbalance comprises a first weight disposed on the balance arm, and a second weight disposed about the first weight, wherein the first weight is movable relative to the balance arm and the second weight is movable relative to the first weight.

10. The system of claim 9, wherein the second weight is adjustable between a distal position and a proximal position on the first weight and moving the second weight between the distal position and proximal position applies a known force to a surface on which the goniolens rests.

11. The system of claim 1, wherein the balance arm is configured to rotate about the pivot point.

12. The system of claim 1, wherein the goniolens comprises a concave surface adapted to contact the patient's eye.

13. The system of claim 12, wherein the goniolens comprises a concave surface that has substantially the same surface geometry as a cornea.

14. The system of claim 1, further comprising a lateral control mechanism configured to control the lateral position of the goniolens.

15. The system of claim 1, further comprising a vertical control mechanism configured to control the vertical position of the goniolens.

16. The system of claim 1, further comprising a rotational control mechanism configured to rotate the balance arm and goniolens.

17. The system of claim 1, further comprising a locking mechanism for the movable counterweight configured to hold the movable counterweight relative to the balance arm.

18. A method of using a gonioscope, the method comprising:
positioning a microscope over a patient's eye so as to put the eye in an optical path of the microscope;
positioning a goniolens in an area adjacent to a patient's eye, the goniolens being attached at one end of a balance arm; and
adjusting a counterweight disposed on the balance arm to contact the goniolens with the patient's eye with a desired contact pressure.

19. The method of claim 18, wherein the counterweight comprises a first weight disposed on the balance arm and a second weight disposed about the first weight, wherein adjusting the counterweight to provide the desired contact pressure on the patient's eye comprises moving the second weight towards the goniolens.

20. The method of claim 18, further comprising viewing the patient's eye through the microscope and goniolens.

21. The method of claim 18, further comprising performing a medical procedure on the patient's eye using two hands.

22. The method of claim 21, wherein the medical procedure is a surgical procedure comprising implantation of a drainage device, laser trabeculoplasty, peripheral laser gonioplasty, goniophotocoagulation, goniotomy, goniosynechialysis, or internal revision of glaucoma filtration operations.

23. The method of claim 21, further comprising moving the microscope to move the position of the goniolens.

24. The method of claim 18, wherein the goniolens is positioned along the optical axis of the microscope.

25. The method of claim 21, wherein performing the medical procedure comprises contacting the patient's eye with the goniolens at two or more positions.

26. An optical system, comprising:
a surgical microscope defining an optical plane;
a balance arm connected to the surgical microscope, the balance arm having a first end, a second end, and a pivot point positioned between the first and second ends;
a movable counterbalance weight disposed on the balance arm between the pivot point and the second end; and
a goniolens disposed on the balance arm towards the first end of the balance arm, the goniolens configured to contact and apply an adjustable force to a patient's eye in the optical plane based on a position of the movable counterbalance.

27. The optical system of claim 26, wherein the counterbalance includes a first weight disposed on the balance arm, and a second weight disposed about the first weight, wherein the first weight is movable relative to the balance arm and the second weight is movable relative to the first weight.

28. The optical system of claim 27, wherein the second weight is adjustable between a distal position and proximal position on the first weight.

29. The optical system of claim 28, wherein moving the second weight between the distal position and proximal position applies a known force on the goniolens.
